(19) United States
(12) Patent Application Publication
(10) Pub. No.: US 2018/0008708 A1
(43) Pub. Date: Jan. 11, 2018

(54) Synchronizing Tumor Cells to the G2/M Phase Using TTFields Combined with Taxane or Other Anti-Microtubule Agents

(71) Applicant: Novocure Limited, St. Helier (JE)

(72) Inventors: Moshe GILADI, Moshav Herut (IL); Tali VOLOSHIN-SELA, Kibbutz Gvat (IL)

(21) Appl. No.: 15/643,578

(22) Filed: Jul. 7, 2017

Related U.S. Application Data
(60) Provisional application No. 62/360,462, filed on Jul. 10, 2016.

(51) Int. Cl. A61K 41/00; A61N 1/32; A61K 31/475; A61N 5/10; A61K 31/337

(52) U.S. Cl. CPC A61K 41/0023; A61N 5/10; A61K 31/337; A61K 31/475; A61N 1/32

(57) ABSTRACT

Cancer cells can be synchronized to the G2/M phase by delivering an anti-microtubule agent (e.g., paclitaxel or another taxane) to the cancer cells, and applying an alternating electric field with a frequency between 100 and 500 kHz to the cancer cells, wherein at least a portion of the applying step is performed simultaneously with at least a portion of the delivering step. This synchronization can be taken advantage of by treating the cancer cells with radiation therapy after the combined action of the delivering step and the applying step has increased a proportion of cancer cells that are in the G2/M phase. The optimal frequency and field strength will depend on the particular type of cancer cell being treated. For certain cancers, this frequency will be between 125 and 250 kHz (e.g., 200 kHz) and the field strength will be at least 1 V/cm.

### CROSS REFERENCE TO RELATED APPLICATIONS

This application claims the benefit of U.S. Provisional Application 62/360,462 filed Jul. 10, 2016, which is incorporated herein by reference in its entirety.

### BACKGROUND

Radiation therapy (RT) is a therapy using ionizing radiation, generally as part of cancer treatment, to control or kill malignant cells. Radiation therapy is often used to treat a number of types of cancer, particularly if they are localized to one area of the body. RT may also be used as part of adjuvant therapy, to prevent tumor recurrence after surgery to remove a primary malignant tumor. RT is often synergistic with chemotherapy, and RT has been used before, during, and after chemotherapy in susceptible cancers.

In vitro experiments demonstrated that radiation therapy is most effective against cells in the G2/M phase of the cell cycle. But because cancer cells are not synchronized in the human body, only a small fraction of cells will exist in the G2/M phase during the course of RT, which can limit treatment efficacy.

Some drugs (e.g., taxanes) have been shown to synchronize cancer cells to the G2/M phase in vitro, and this leads to increased efficacy of subsequent RT. Still, while this process was successfully shown in vitro, its applicability in vivo remains controversial in part because the pharmacokinetics and pharmacodynamics of taxanes often result in low concentrations in a tumor which are insufficient to achieve significant synchronization in vivo.

### SUMMARY OF THE INVENTION

One aspect of the invention is directed to a first method of killing cancer cells. This method comprises delivering a taxane to the cancer cells and applying an alternating electric field to the cancer cells. The alternating electric field has a frequency between 100 and 500 kHz, and at least a portion of the applying step is performed simultaneously with at least a portion of the delivering step. This method also comprises treating the cancer cells with a radiation therapy after a combined action of the delivering step and the applying step has increased a proportion of cancer cells that are in the G2/M phase.

In some embodiments of the first method, the taxane comprises paclitaxel. In some of these embodiments, the paclitaxel is delivered to the cancer cells at a concentration of less than 10 nM.

In some embodiments of the first method, the alternating electric field has a field strength of at least 1 V/cm in at least some of the cancer cells, and a frequency between 125 and 250 kHz.

In some embodiments of the first method, the treating step is performed after the combined action of the delivering step and the applying step has increased a proportion of cancer cells that are in the G2/M phase to at least 50%.

In some embodiments of the first method, the treating step is performed after the applying step has ended. In some embodiments of the first method, the treating step is performed while the applying step is ongoing. In some embodiments of the first method, the treating step is performed after at least eight hours of the applying step have elapsed.

Another aspect of the invention is directed to a second method of synchronizing cancer cells to a G2/M phase. This method comprises delivering an anti-microtubule agent to the cancer cells, and applying an alternating electric field to the cancer cells. The alternating electric field has a frequency between 100 and 500 kHz, and at least a portion of the applying step is performed simultaneously with at least a portion of the delivering step.

In some embodiments of the second method, the anti-microtubule agent comprises paclitaxel. In some embodiments of the second method, the anti-microtubule agent comprises a taxane. In some embodiments of the second method, the anti-microtubule agent comprises vincristine. In some embodiments of the second method, the anti-microtubule agent comprises a vinca alkaloid.

In some embodiments of the second method, the combination of the delivering step and the applying step results in a cell distribution with at least 50% of the cancer cells in the G2/M phase.

In some embodiments of the second method, the alternating electric field has a field strength of at least 1 V/cm in at least some of the cancer cells, and a frequency between 125 and 250 kHz.

Some embodiments of the second method further comprise treating the cancer cells with radiation therapy after a combined action of the delivering step and the applying step has increased a proportion of cancer cells that are in the G2/M phase. In some of these embodiments, the treating step is performed after the combined action of the delivering step and the applying step has increased a proportion of cancer cells that are in the G2/M phase to at least 50%. In these embodiments, the treating step may be performed after the applying step has ended, or while the applying step is ongoing. In these embodiments, the treating step may be performed after at least eight hours of the applying step have elapsed.

### BRIEF DESCRIPTION OF THE DRAWINGS

FIGS. 1A-1H depict cell cycle distributions following 72 hours of different treatments at various doses with and without TTFields for OVCAR-3 cells.

FIG. 2A is a set of bar graphs that represents the change in percentage of A2780 cells in the G2/M phase following treatment.

FIG. 2B is a set of bar graphs that represents the change in percentage of OVCAR-3 cells in the G2/M phase following treatment.

FIG. 2C is a set of bar graphs that represents the change in percentage of Caov-3 cells in the G2/M phase following treatment.

FIGS. 3A-3D depict images of mitotic figures for the A2780 cell line obtained using confocal microscopy after four different courses of treatment.

FIGS. 4A-4D depict images of mitotic figures for the OVCAR-3 cell line obtained using confocal microscopy after four different courses of treatment.

FIGS. 5A-5D depict images of mitotic figures for the Caov-3 cell line obtained using confocal microscopy after four different courses of treatment.

Various embodiments are described in detail below with reference to the accompanying drawings, wherein like reference numerals represent like elements.

### DESCRIPTION OF THE PREFERRED EMBODIMENTS

Tumor Treating Fields (TTFields) are low intensity, intermediate frequency alternating electric fields that target solid tumors by disrupting mitosis. TTFields are preferably delivered through two pairs of transducer arrays positioned to generate electric fields in the tumor in two different directions in an alternating sequence. Although these two different directions are preferably as close to perpendicular as possible, exact perpendicularity is not required. TTFields are approved by the FDA for the treatment of Glioblastoma, and clinical trials testing the efficacy of TTFields for various solid tumors are underway.

The in vitro experiments described below demonstrated that applying TTFields alone (i.e., without a taxane such as paclitaxel) resulted in a small increase in the percentile of OVCAR-3 cells in the G2/M phase, but no significant change in the percentile of Caov-3 and A2780 cells in the G2/M phase. Based on these experiments, the inventors do not expect TTFields at those field strengths and frequencies, when used alone, to be particularly useful for synchronizing tumor cells to the G2/M phase. But surprisingly, when the delivery of low dose taxanes was combined with the application of TTFields, the combination was a very effective tool for synchronizing tumor cells into the G2/M phase. Because RT is most effective against cells in the G2/M phase of the cell cycle, this combination is useful for sensitizing the cells to RT prior to any given session of RT. After sensitization occurs, treatment using RT can then proceed using a conventional RT protocol. And due to the enhanced sensitization to RT provided by the combination of the TTFields and the taxane, the effectiveness of the conventional RT treatment will be enhanced.

Below we discuss sensitizing tumor cells to radiation therapy by synchronizing the cells to the G2/M phase using a combination of both TTFields and low dose taxanes.

Note that although the example discussed herein uses paclitaxel in combination with TTFields to synchronize the cells, in alternative embodiments other taxanes or other low-dose anti microtubule agents (e.g., Vincristine or another vinca alkaloid) may be used in place of paclitaxel. Note also that while the experimental results described herein were obtained in vitro, the inventors expect that they will carry over to the in vivo context.

In some embodiments, the anti-microtubule agents are delivered in low dose concentrations continuously to coincide with the exposure to TTFields. In some embodiments, the TTFields are delivered to tumors/organs in which there is a low permeability of anti-microtubule agents (e.g., the brain) and the drug is delivered by administering it systemically. In some embodiments, the drug is delivered by administering it locally.

In some embodiments, the radiation therapy is applied immediately after TTFields application is stopped and the electrode arrays (which are used to apply the TTFields) are removed. In some embodiments, the radiation therapy is applied through the arrays. In some embodiments, other radio sensitizing agents are added to the treatment. In some embodiments, RT is delivered according to the standard protocol for the treatment of GBM patients (e.g., five fractions of 2 Gy delivered on Monday through Friday) and TTFields are applied between the cycles of RT (e.g., during the weekend) in combination with anti microtubule agents which can penetrate the blood brain barrier even in a low dose. In some embodiments, the TTFields are applied in combination with anti microtubule agents before and after each RT treatment.

Proof of concept was established in the experiments described below.

Cell Culture and Drugs

The human ovarian carcinoma cell line A2780 was obtained from the European Collection of Cell Cultures. The human ovarian adenocarcinoma cell lines OVCAR-3 (HTB-161) and Caov-3 (HTB-75) were obtained from the American Type Culture Collection (ATCC). Paclitaxel (Sigma Aldrich, Rehovot, Israel) dissolved in DMSO was used at the following concentrations: 1 nM, 2 nM, 4 nM, 10 nM, and 100 nM.

TTFields Application in Vitro

TTFields were applied in vitro using special ceramic Petri dishes with two pairs of transducer arrays printed perpendicularly on the outer walls of a Petri dish. The inner surfaces of the electrodes were coated with a high dielectric constant ceramic (lead magnesium niobate-lead titanate (PMN-PT)). The transducer arrays were connected to a sinusoidal waveform generator which generated fields at 200 kHz in the medium. By selectively activating the signals that were applied to the electrodes, the orientation of the TTFields was switched 90° every 1 second, thus covering the majority of the orientation axis of cell divisions, as previously described by Kirson et al. During the experiment, temperature was measured by 2 thermistors (Omega Engineering, Stamford, Conn.) attached to the walls of the Petri dish. All cells suspensions were grown on a cover slip inside the Petri dish and treated with TTFields at intensity of 2.7 V/cm. TTFields were applied for 8-72 hours alone or in combination with different dosages of paclitaxel. Those same dosages (including the zero dosage) were also tested without the application of TTFields.

Flow Cytometry

For cell cycle analysis, cells were washed twice with PBS and fixed with 70% ice cold ethanol for 30 minutes. After fixation cells were pelleted and incubated in PBS containing 10 μg/ml RNase and 7.5 μg/ml 7-AAD at 37° C. for 30 minutes. Cell cycle distribution was then quantified using iCyt EC800. Fluorescence signals were collected at the wavelengths of 525/50 nm for Annexin V and 665/30 nm for 7-AAD. The data was analyzed using the Flowjo software.

Microscopy

For mitotic figures analysis, cells were grown on glass cover slips and treated using the ceramic Petri dish system described above for either 8 or 72 hours. At the end of the experiment, cells were fixed with ice cold methanol for 10 minutes. The cells were then serum-blocked, and stained with rabbit anti-human α-tubulin antibodies (Abcam) for 2 hours. Alexa Fluor 488-conjugated secondary antibody was used (Jackson ImmunoResearch). DNA was stained with the dye 4′,6-diamidino-2-phenylindole (DAPI) (Sigma-Aldrich) at 0.2 μg/ml for 20 min. Images were collected using a LSM 700 laser scanning confocal system, attached to an upright motorized microscope with ×20 and ×63/1.40 oil objective (ZeissAxio Imager Z2).

Results

To assess whether adding TTFields to paclitaxel affects the responsiveness of ovarian carcinoma cells, we treated the cells with paclitaxel alone at different dosages and also at a zero dosage. We also treated the cells at those same dosages in combination with TTFields (2.7 V/cm pk-pk, 200 kHz). Flow cytometry was used to measure the results.

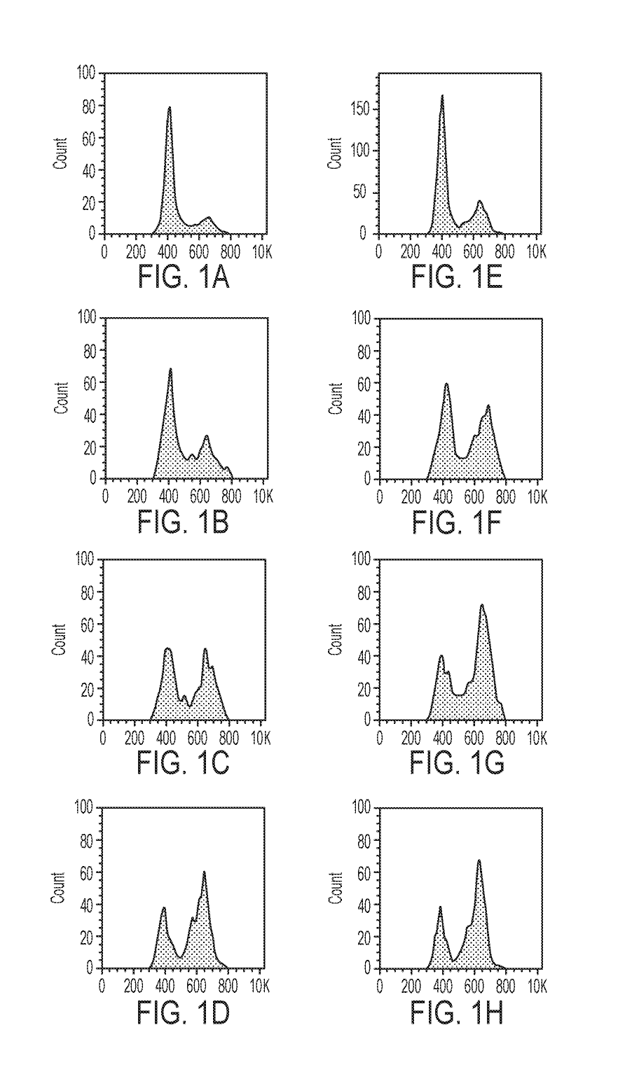

FIGS. 1A-1H are representative plots of cell cycle distributions following 72 hours of the different treatments at various doses with and without TTFields for OVCAR-3 cells. More specifically: FIG. 1A depicts the cell cycle distribution for a control sample in which no paclitaxel was administered and TTFields were not applied; FIG. 1E depicts the cell cycle distribution when no paclitaxel was administered and TTFields were applied; FIGS. 1B, 1C, and 1D depict the cell cycle distributions for samples in which paclitaxel was delivered at concentrations of 2, 4, and 100 nM, respectively, and TTFields were not applied; and FIGS. 1F, 1G, and 1H depict the cell cycle distributions for samples in which paclitaxel was delivered at concentrations of 2, 4, and 100 nM, respectively, and TTFields were applied. Note that the peaks on the right half of each panel of FIGS. 1A-1H represent the G2/M phase fraction.

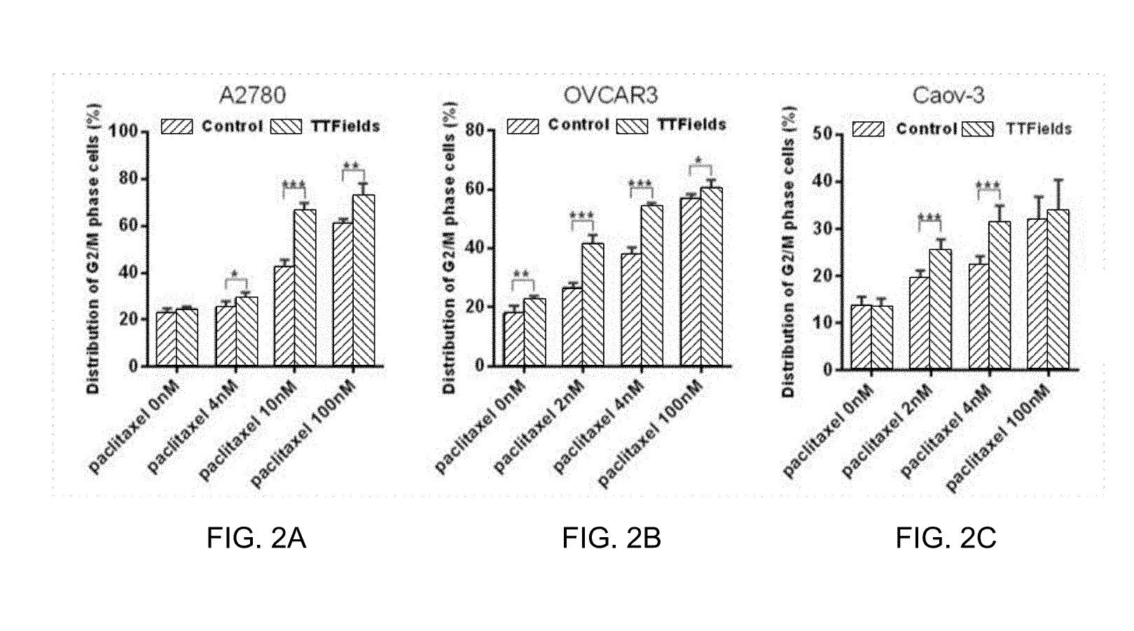

FIG. 2A is a set of bar graphs that represents the change in percentage of A2780 cells in the G2/M phase following treatment for 8 hours at various doses with and without TTFields. FIG. 2B is a set of bar graphs that represents the change in percentage of OVCAR-3 cells in the G2/M phase following treatment for 72 hours at various doses with and without TTFields. FIG. 2C is a set of bar graphs that represents the change in percentage of Caov-3 cells in the G2/M phase following treatment for 72 hours at various doses with and without TTFields. Note that in FIGS. 2A-2C, the left half of each pair of bars is without TTFields, and the right half of each pair is with TTFields.

The Flow cytometry revealed that cells exposed to paclitaxel alone were blocked in cell cycle progression and accumulated in the G2/M phase in a dose dependent manner. This is apparent from FIGS. 1A-1D and the left half of each pair of bars in FIGS. 2A-2C.)

Applying TTFields alone (paclitaxel 0 nM) resulted in a statistically significant but minor increase in the percentile of OVCAR-3 cells in the G2/M phase (this is apparent from a comparison of FIG. 1A with FIG. 1E, and also from the 0 nM pair of bars in FIG. 2B) and no significant change in the percentile of Caov-3 and A2780 cells in the G2/M phase (see the 0 nM pair of bars in FIGS. 2A and 2C).

But surprisingly, 72 hours simultaneous treatment with low dose paclitaxel (2, 4 and 10 nM) combined with TTFields dramatically increased the number of Caov-3 and OVCAR-3 cells in the G2/M phase of the cell cycle (this is apparent from a comparison of FIG. 1B with FIG. 1F, from a comparison of FIG. 1C with FIG. 1G, and from FIGS. 2B and 2C). In addition, as seen in FIG. 2A, A2780 cells exposed to the combination of low dose paclitaxel and TTFields accumulated in the G2/M phase even after a short treatment duration (8 hours).

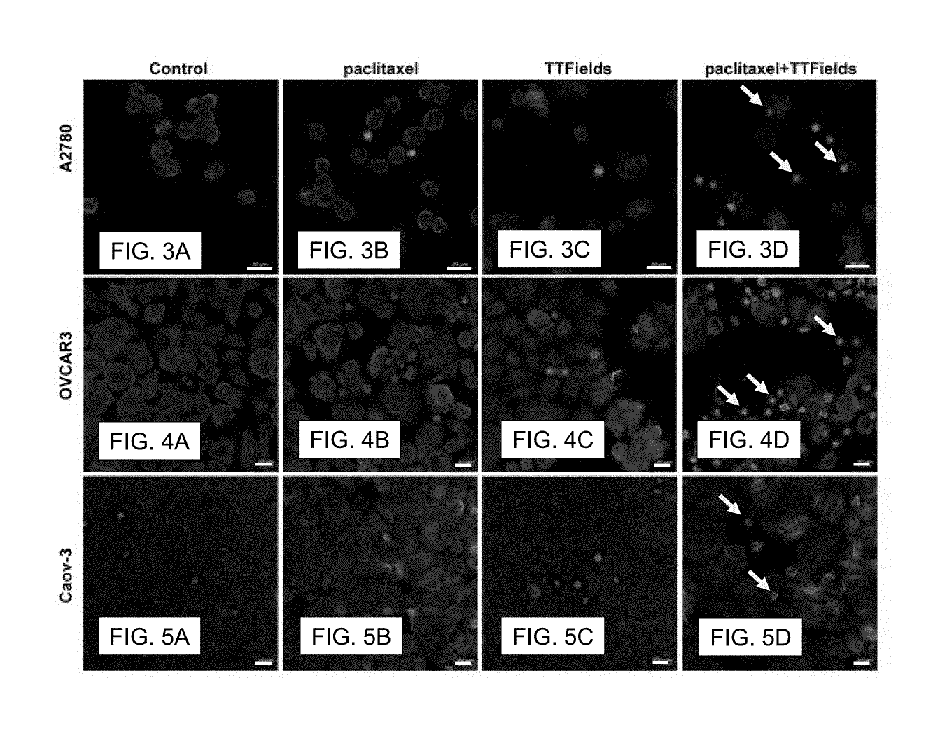

To verify these effects observed by flow cytometry, we examined the appearance of mitotic figures following 72 hours of different treatments using confocal microscopy. FIGS. 3A-3D depict these results for a control (FIG. 3A); 4 nM paclitaxel alone (FIG. 3B); 2.7 V/cm pk-pk, 200 kHz TTFields alone (FIG. 3C); and 4 nM paclitaxel combined with 2.7 V/cm pk-pk, 200 kHz TTFields (FIG. 3D) for the A2780 cell line. FIGS. 4A-4D depict these results for a control (FIG. 4A); 4 nM paclitaxel alone (FIG. 4B); 2.7 V/cm pk-pk, 200 kHz TTFields alone (FIG. 4C); and 4 nM paclitaxel combined with 2.7 V/cm pk-pk, 200 kHz TTFields (FIG. 4D) for the OVCAR-3 cell line. FIGS. 5A-5D depict these results for a control (FIG. 5A); 4 nM paclitaxel alone (FIG. 5B); 2.7 V/cm pk-pk, 200 kHz TTFields alone (FIG. 5C); and 4 nM paclitaxel combined with 2.7 V/cm pk-pk, 200 kHz TTFields (FIG. 5D) for the Caov-3 cell line. The scale bar (which is the small white line on the bottom right of each of FIGS. 3A-5D) represents 20 μm.

In all three cell lines tested, combination treatment with TTFields and low dose paclitaxel (FIGS. 3D, 4D, and 5D) displayed a substantial increase in mitotic figures, indicative of mitotic arrest, as compared to the other treatments (FIGS. 3A-C, FIGS. 4A-C, and FIGS. 5A-C). The arrows in FIGS. 3D, 4D, and 5D indicate representative mitotic figures.

Taken together, these results provide further evidence for the strong synergy between paclitaxel and TTFields in the treatment of ovarian cancer cells. We expect this synergy will be present for other types of cancer cells as well.

These results establish that cancer cells can be synchronized to the G2/M phase by delivering an anti-microtubule agent to the cancer cells, and applying an alternating electric field with a frequency between 100 and 500 kHz to the cancer cells, wherein at least a portion of the applying step is performed simultaneously with at least a portion of the delivering step. Examples of anti-microtubule agents that may be used for this purpose include taxanes (e.g., paclitaxel) and vinca alkaloids (e.g., vincristine). The combination of the delivering step and the applying step can be used to obtain a cell distribution with at least 50% of the cancer cells in the G2/M phase. The optimal frequency and field strength will depend on the particular type of cancer cell being treated. For certain cancers, this frequency will be between 125 and 250 kHz (e.g., 200 kHz) and the field strength will be at least 1 V/cm.

The synchronization described in the previous paragraph can be taken advantage of by treating the cancer cells with radiation therapy after the combined action of the delivering step and the applying step (as described in the previous paragraph) has increased a proportion of cancer cells that are in the G2/M phase. For example, the RT may be performed after the combined action of the delivering step and the applying step has increased a proportion of cancer cells that are in the G2/M phase to at least 50%. The RT may be performed after the applying step has ended or while the applying step is ongoing. The RT may be performed after at least eight hours of the applying step have elapsed.

It follows that cancer cells can be killed by delivering a taxane to the cancer cells and applying an alternating electric field with a frequency between 100 and 500 kHz to the cancer cells, wherein at least a portion of the applying step is performed simultaneously with at least a portion of the delivering step. After a combined action of the delivering step and the applying step has increased a proportion of cancer cells that are in the G2/M phase, the cancer cells are treated with RT. For example, the RT may be performed after the combined action of the delivering step and the applying step has increased a proportion of cancer cells that are in the G2/M phase to at least 50%. The RT may be performed after the applying step has ended or while the applying step is ongoing. The RT may be performed after at least eight hours of the applying step have elapsed.

One example of a suitable taxane is paclitaxel, which may be delivered to the cancer cells at a concentration of less than 10 nM. The optimal frequency and field strength will depend on the particular type of cancer cell being treated. For certain cancers, this frequency will be between 125 and 250 kHz (e.g., 200 kHz) and the field strength will be at least 1 V/cm.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method of killing cancer cells, the method comprising:
delivering a taxane to the cancer cells; and
applying an alternating electric field to the cancer cells, the alternating electric field having a frequency between 100 and 500 kHz, wherein at least a portion of the applying step is performed simultaneously with at least a portion of the delivering step; and
treating the cancer cells with a radiation therapy after a combined action of the delivering step and the applying step has increased a proportion of cancer cells that are in the G2/M phase.

2. The method of claim 1, wherein the taxane comprises paclitaxel.

3. The method of claim 1, wherein the taxane comprises paclitaxel, and wherein the paclitaxel is delivered to the cancer cells at a concentration of less than 10 nM.

4. The method of claim 1, wherein the alternating electric field has a field strength of at least 1 V/cm in at least some of the cancer cells, and a frequency between 125 and 250 kHz.

5. The method of claim 1, wherein the treating step is performed after the combined action of the delivering step and the applying step has increased a proportion of cancer cells that are in the G2/M phase to at least 50%.

6. The method of claim 1, wherein the treating step is performed after the applying step has ended.

7. The method of claim 1, wherein the treating step is performed while the applying step is ongoing.

8. The method of claim 1, wherein the treating step is performed after at least eight hours of the applying step have elapsed.

9. A method of synchronizing cancer cells to a G2/M phase, the method comprising:
delivering an anti-microtubule agent to the cancer cells; and
applying an alternating electric field to the cancer cells, the alternating electric field having a frequency between 100 and 500 kHz,
wherein at least a portion of the applying step is performed simultaneously with at least a portion of the delivering step.

10. The method of claim 9, wherein the anti-microtubule agent comprises paclitaxel.

11. The method of claim 9, wherein the anti-microtubule agent comprises a taxane.

12. The method of claim 9, wherein the anti-microtubule agent comprises vincristine.

13. The method of claim 9, wherein the anti-microtubule agent comprises a vinca alkaloid.

14. The method of claim 9, wherein the combination of the delivering step and the applying step results in a cell distribution with at least 50% of the cancer cells in the G2/M phase.

15. The method of claim 9, wherein the alternating electric field has a field strength of at least 1 V/cm in at least some of the cancer cells, and a frequency between 125 and 250 kHz.

16. The method of claim 9, further comprising treating the cancer cells with radiation therapy after a combined action of the delivering step and the applying step has increased a proportion of cancer cells that are in the G2/M phase.

17. The method of claim 16, wherein the treating step is performed after the combined action of the delivering step and the applying step has increased a proportion of cancer cells that are in the G2/M phase to at least 50%.

18. The method of claim 16, wherein the treating step is performed after the applying step has ended.

19. The method of claim 16, wherein the treating step is performed while the applying step is ongoing.

20. The method of claim 16, wherein the treating step is performed after at least eight hours of the applying step have elapsed.
